(19) United States
(12) Patent Application Publication
(10) Pub. No.: US 2004/0024180 A1
(43) Pub. Date: Feb. 5, 2004

(54) Process for the production of 2,5 -diketopiperazines,2,5-diketopiperazines , dipeptides and their use thereof

(76) Inventors: Karlheinz Drauz, Freigericht (DE); Gunter Knaup, Bruchkobel (DE)

Correspondence Address: Michael A Sanzo, Fitch Even Tabin & Flannery, 1801 K Street N W, Suite 401L, Washington, DC, 20006-1201, US

(21) Appl. No.: 10/258,029

(22) PCT Filed: Mar. 23, 2001
(86) PCT No.: PCT/EP01/03322

(30) Foreign Application Priority Data
Apr. 20, 2000 (DE) 10019879.1

(51) Int. Cl. C07K 5/12; C07K 5/06

(52) U.S. Cl. 530/331

(57) ABSTRACT

Process for the production of 2,5-diketopiperazines of general formula I,

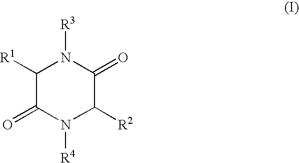

by heating dipeptides of general formula II

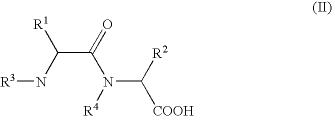

in an organic solvent whilst removing water by distillation.

Novel 2,5-diketopiperazines, dipeptides and their use.

The present invention relates to a process for the production of 2,5-diketopiperazines of general formula I,

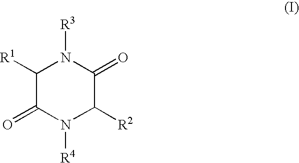

in which R¹, R² independently of each other represent H, (C₁-C₈)-alkyl, (C₂-C₈)-alkenyl, (C₂-C₈)-alkinyl, (C₁-C₈)-alkoxy, (C₃-C₈)-cycloalkyl, (C₆-C₁₈)-aryl, (C₇-C₁₉)-aralkyl, (C₃-C₁₈)-heteroaryl, (C₄-C₁₉)-heteroaralkyl, ((C₁-C₈)-alkyl)₁₋₃-(C₃-C₈)-cycloalkyl, ((C₁-C₈)-alkyl)₁₋₃-(C₆-C₁₈)-aryl, ((C₁-C₈)-alkyl)₁₋₃-(C₃-C₁₈)-heteroaryl, or the side chain group of an α-amino acid,

R³, R⁴ independently of each other represent H, (C₁-C₈)-alkyl, (C₂-C₈)-alkenyl, (C₂-C₈)-alkinyl, (C₁-C₈)-acyl, (C₃-C₈)-cycloalkyl, (C₆-C₁₈)-aryl, (C₇-C₁₉)-aralkyl, (C₃-C₁₈) heteroaryl, (C₄-C₁₉)-heteroaralkyl, ((C₁-C₈)-alkyl)₁₋₃-(C₃-C₈)-cycloalkyl, ((C₁-C₈)-alkyl)₁₋₃-(C₆-C₁₈)-aryl, ((C₁-C₈)-alkyl)₁₋₃-(C₃-C₁₈)-heteroaryl, or

R¹ and R³ and/or R² and R⁴ form a ring via a (C₂-C₈)-alkylene unit and also the use of the compounds of formula I produced by such a process.

A further aspect of the invention concerns special 2,5-diketopiperazines, dipeptides and their use. 2,5-diketopiperazines, i.e. cyclic dipeptides, are a class of substances found widely in nature (F. T. Witiak, Y. Wei, Prog. Drug. Res. 35, 249 (1990)). In most cases, they are formed by the decomposition of proteins and are used as flavourings in many foodstuffs such as e.g. beer (M. Gautschiet, J. Agri. Food Chem. 45, 3183 (1997)). A number of diketopiperazines, such as e.g. cyclo[Pro-His] also have a pharmacological action (U.S. Pat. No. 5,418,218). Structures derived from diketopiperazines are being developed as pharmaceutical products (e.g. U.S. Pat. No. 5,932,579) or are already in use as such (e.g. dihydroergotoxin, A. Stoll, Helv. Chim. Acta 26, 2070 (1943), DOS 2802113). They are also used as Drug Delivery Systems (WO 9610396, WO 9609813, U.S. Pat. No. 5,503,852, WO 9318754).

Diketopiperazines can also be used as chiral catalysts, e.g. for the production of chiral cyanohydrines (M. North, Synlett, 1993, 807) or as educts for enantio-selective production of amino acids (U. Schollkopf, Tetrahedron 39, 2085 (1983)).

The most common method of producing 2,5-diketopiperazines is to release esters of the corresponding dipeptides from the salts and optionally to heat them (E. Fischer, Chem. Ber. 34, 2893 (1903)). However, as the free esters are basic and, on the other hand, it is known that diketopiperazines racemise more easily than the corresponding dipeptides or amino acids, the possibility of partial racemisation must always be considered with this method. This can largely be avoided by adding acetic acid when cyclising the esters (T. Ueda, Bull. Chem. Soc. Jpn., 50 566 (1983). Nevertheless, this method has the disadvantage that the esters must first be produced from the dipeptides or an amino acid ester must be used to produce the dipeptides. In both cases, an additional process step is required.

Some 2,5-diketopiperazines can also be obtained by heating the dipeptides in water to temperatures of >100° C. (S. Steinberg, Science 213, 544 (1981)). However, as diketopiperazines are relatively easily hydrolysed, full conversion cannot be achieved by this method. Rather, an equilibrium is established between the diketopiperazine and the two dipeptides.

The object was therefore to provide another process for the production of 2,5-diketopiperazines, which makes it possible to produce sufficient yields of the desired compounds with a good degree of purity. In particular, the process should be suitable for use on an industrial scale, i.e. it should be possible to generate the 2,5-diketopiperazines by the most economically and ecologically advantageous means.

This object is achieved by a process according to claim 1. Claims 2 to 6 represent preferred embodiments of the process according to the invention. Claims 7 to 10 protect special 2,5-diketopiperazines and their precursors, the dipeptides. Claims 11 and 12 focus on preferred uses.

By using a process for the production of 2,5-diketopiperazines of general formula I,

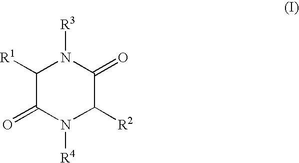

in which R¹, R² independently of each other represent H, (C₁-C₈)-alkyl, (C₂-C₈)-alkenyl, (C₂-C₈)-alkinyl, (C₁-C₈)-alkoxy, (C₃-C₈)-cycloalkyl, (C₆-C₁₈)-aryl, (C₇-C₁₉)-aralkyl, (C₃-C₁₈)-heteroaryl, (C₄-C₁₉)-heteroaralkyl, ((C₁-C₈)-alkyl)₁₋₃-(C₃-C₈)-cycloalkyl, ((C₁-C₈)-alkyl)₁₋₃-(C₆-C₁₈)-aryl, ((C₁-C₈)-alkyl)₁₋₃-(C₃-C₁₈)-heteroaryl, or the side chain groups of an α-amino acid R³, R⁴ independently of each other represent H, (C₁-C₈)-alkyl, (C₂-C₈)-alkenyl, (C₂-C₈)-alkinyl, (C₁-C₈)-acyl, (C₃-C₈)-cycloalkyl, (C₆-C₁₈)-aryl, (C₇-C₁₉)-aralkyl, (C₃-C₁₈)-heteroaryl, (C₄-C₁₉)-heteroaralkyl, ((C₁-C₈)-alkyl)₁₋₃-(C₃-C₈)-cycloalkyl, ((C₁-C₈)-alkyl)₁₋₃-(C₆-C₁₈)-aryl, ((C₁-C₈)-alkyl)₁₋₃-(C₃-C₁₈)-heteroaryl, or

R¹ and R³ and/or R² and R⁴ form a ring via a (C₂-C₈)-alkylene unit,

in which dipeptides of general formula II

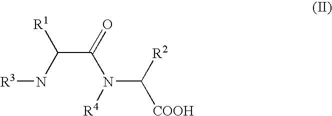

in which R¹, R², R³, R⁴ have the meaning given above, are heated in an organic solvent whilst removing water by distillation, a process that can be carried out advantageously on an industrial scale with good yields of the desired 2,5-diketopiperazines at a high degree of purity is achieved surprisingly easily. The piperazines are partly obtained in a crystallisation yield of up to 70% with a purity of >99% per HPLC after one crystallisation, in particular highly enantiomer-enriched.

In principle, all organic solvents can be considered as a solvent, that are capable of removing sufficient quantities of water from the reaction mixture at increased temperatures. Solvents that form a low-boiling azeotrope with water, such as e.g acetonitrile, allyl alcohol, benzene, benzyl alcohol, n-butanol, 2-butanol, tert.-butanol, acetic acid butylester, carbon tetrachloride, chlorobenzene, chloroform, cyclohexane, 1,2-dichlorethane, diethylacetal, dimethylacetal, acetic acid ethylester, heptane, methylisobutylketone, 3-pentanol, toluene, xylene, are preferred in particuler. n-butanol is preferred most particularly as a solvent.

The temperature of the reaction depends firstly on the reaction speed at which the cyclisation takes place and secondly on the type of azeotroping agent used. It is also restricted by the cost factor of the energy to be used. The reaction is preferably carried out at 50-200° C., in particular at 80-150° C. The pH range in which cyclisation takes place can easily be determined by the person skilled in the art, in principle by means of routine experiments. It is advantageously 2 to 9, preferably 3 to 7.

With regard to the use of synthesis on an industrial scale, it is particularly advantageous if the dipeptides of formula (II) can be used in the cyclising reaction in the form of an aqueous solution. This variant is advantageously used if hydrolysable protective groups such as e.g. Ncarboxylic acid anhydride, tert.-butyloxycarbonyl-, formyl- or fluourenylmethoxycarbonyl are used as an N-terminal protective group for peptide coupling. In these cases the protective groups can be split off without isolation, directly in the reaction solution to be used for cyclisation. Nor do the bases required in most cases for coupling with free amino acids, such as e.g. alkali hydroxides or -carbonates, tert.-amines, have to be split off; they can remain in the solution after neutralisation in the form of their salts.

As the 2,5-diketopiperazines are generally considerably less soluble in water than the corresponding dipeptides, they can simply be purified after the reaction has taken place by treatment with water, all salts and optionally unreacted dipeptides or amino acids being removed. In cases in which the 2,5-diketopiperazines are soluble in organic, non water-miscible solvents, this purification can even be carried out by extraction with water.

The advantages of the process according to the invention are demonstrated impressively by a reference example. Whilst the cyclisation of an aqueous L-phenylalanyl-L-prolin solution at pH 4 with n-Butanol delivers a conversion of 99% after just one hour, when heating the same solution to reflux temperature without n-butanol, only 19% conversion is achieved after 4 hours. Although, after 20 hours at this temperature, the L-phenylalanyl-L-prolin is no longer detectable, 30% of the inverse dipeptide L-prolyl-L-phenylalanine is obtained alongside 70% of the 2,5-diketopiperazine. This is not obtained in the process according to the invention.

In a further development the invention relates to 2,5-diketopiperazines of general formula III,

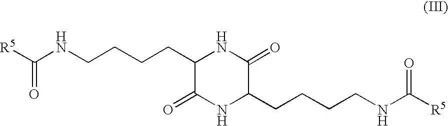

in which R⁵ represents H or trifluoromethyl. The (S,S) configuration of this compound is preferred.

The invention also relates to dipeptides of formula

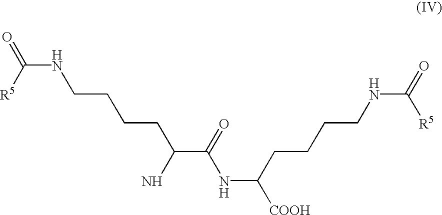

in which R⁵ represents H or trifluoromethyl. The (S,S) configuration of this compound is also preferred. III and IV are preferably used to produce cyclo[Lys-Lys]. The compounds of formula I according to the invention can be used in the synthesis of bio-active compounds.

Methyl, ethyl, n-Propyl, isopropyl, n-Butyl, isobutyl, sec-butyl, tert-butyl, pentyl, hexyl, heptyl or octyl and all bond isomers are to be considered as (C₁-C₈)-alkyl. These can be mono- or poly-substituted with (C₁-C₈)-alkoxy, (C₁-C₈)-haloalkyl, OH, halogen, NH₂, NO₂, SH, S-(C₁-C₈)alkyl.

(C₂-C₈)-alkenyl, with the exception of methyl, is understood to mean a (C₁-C₈)-alkyl group as illustrated above having at least one double bond.

(C₂-C₈)-alkinyl, with the exception of methyl, is understood to mean a (C₁-C₈)-alkyl group as illustrated above, having at least one triple bond.

(C₁-C₈)-acyl is understood to mean a (C₁-C₈)-alkyl group bound to the molecule by a C═O function.

(C₃-C₈)-Cycloalkyl is understood to mean cyclopropyl, cyclobutyl, cyclopentyl, cyclohexyl or cycloheptyl groups etc. These may be substituted with one or more halogens and/or groups containing N—, O—, P—, S-atoms- and/or may have groups containing N—, O—, P—, S-atoms- in the ring, such as e.g. 1-, 2-, 3-, 4-piperidyl, 1-, 2-, 3-pyrrolidinyl, 2-, 3-tetrahydrofuryl, 2-, 3-, 4-morpholinyl. These can also be mono- or poly-substituted with (C₁-C₈)-alkoxy, (C₁-C₈)-haloalkyl, OH, C₁, NH₂, NO₂.

A (C₆-C₁₈)-aryl group is understood to be an aromatic group with 6 to 18 C-atoms. These include in particular compounds such as phenyl-, naphthyl-, anthryl-, phenanthryl-, biphenyl groups. It can be mono-or poly-substituted with (C₁-C₈)-alkoxy, (C₁-C₈)-haloalkyl, OH, halogen, NH₂, NO₂, SH, S—(C₁-C₈)-alkyl.

A (C₇-C₁₉)-aralkyl group is a (C₆-C₁₈)-aryl group bound to the molecule by a (C₁-C₈)-alkyl group.

(C₁-C₈)-alkoxy is a (C₁-C₈)-alkyl group bound to the molecule under consideration by an oxygen atom.

(C₁-C₈)-haloalkyl is a (C₁-C₈)-alkyl group substituted with one or more halogen atoms.

A (C₃-C₁₈)-heteroaryl group means, in the context of the invention, a five-, six-, or seven-link aromatic ring system of 3 to 18 C atoms, which has heteroatoms such as nitrogen, oxygen or sulfur in the ring. Groups such as 1-, 2-, 3-furyl, such as 1-, 2-, 3-pyrrolyl, 1-, 2-,3-thienyl, 2-, 3-, 4-pyridyl, 2-, 3-, 4-, 5-, 6-, 7-indolyl, 3-, 4-, 5-pyrazolyl, 2-, 4-, 5-imidazolyl, acridinyl, chinolinyl, phenanthridinyl, 2-, 4-, 5-, 6-pyrimidinyl are considered in particular to be such heteroatoms. It can be mono-or poly-substituted with (C₁-C₈)-alkoxy, (C₁-C₈)-haloalkyl, OH, halogen, NH₂, NO₂, SH, S-(C₁-C₈)-alkyl.

A (C₄-C₁₉)-heteroaralkyl is understood to be a heteroaromatic system corresponding to the (C₇-C₁₉)-aralkyl group.

The term (C₁-C₈)-alkylene unit is understood to mean a (C₁-C₈)-alkyl group, which is bound to the relevant molecule by two of its C atoms. It can be mono- or poly-substituted with (C₁-C₈)-alkoxy, (C₁-C₈)-haloalkyl, OH, halogen, NH₂, NO₂, SH, S-(C₁-C₈)-alkyl.

Fluorine, chlorine, bromine and iodine may be considered as halogens.

A side-chain group of an α-amino acid is understood to mean the changeable group on the α-C atom of glycine as the basic amino acid. Natural 1-amino acids are given for example in Bayer-Walter, Lehrbuch der organischen Chemie, S. Hirzel Verlag, Stuttgart, 22nd edition, page 822ff.

Preferred synthetic α-amino acids are those from DE 19903268.8. The side chain groups can be derived from those referred to there.

The stated chemical structures relate to all possible stereoisomers that can be obtained by varying the configuration of the individual chiral centres, axes or surfaces, in other words all possible diastereomers as well as all optical isomers (enantiomers) falling within this group.

In the context of the invention the term enantiomer-enriched is understood to mean the content of an enantiomer in the mixture with its optical antipodes in a range of >50% and <100%.

### EXAMPLES

Production of Cyclo [L-phenylalanyl-L-prolyl]

a. Cyclisation at pH=6.4

1940 g of an aqueous solution of 235 g L-phenylalanyl-L-proline, which still contained 7 g L-phenylalanine and ca 300 g potassium chloride was set to pH 6.4 and concentrated in a vacuum to a thick crystal paste. 1 1 n-butanol was then added and the mixture was heated for 2 hours in the water separator. According to HPLC the mixture then consisted of 57% DKP and 26% dipeptide. After cooling, 700 ml water was added and the phases were separated. The organic phase was washed again with 150 ml water and concentrated in a vacuum. The remaining oil was stirred up with MTBE and the solid formed was filtered off. 113 g (52% of theoretical) cyclo[L-phenylalanyl-L-prolyl] with an HPLC-purity of >99% and an [α]_{D/20} of −105.1° (c=1, n-butanol) was obtained.

b. Cyclisation at pH=4.0

100 ml of the aqueous dipeptide solution used in example 1a was set to pH 4.0 and reacted as in example 1a. After heating for 1 hour, the ratio of DKP.dipeptide was 99:1.

c. Cyclisation at pH=4.0 in Water

100 ml of the aqueous dipeptide solution used in example 1a was set to pH 4.0 and heated to boiling point. The course of the reaction was followed by HPLC. The ratio of DKP dipeptide was 19:81 after 2 hours and 39:61 after 4 hours. After 24 hours no more L-phenylalanyl-L-prolin was detectable. Instead, the DKP and the L-prolyl-L-phenylalanine was detected in a ratio of 69:31.

Production of Cyclo[L-valyl-L-prolyl]

1740 g of an aqueous solution of 132 g L-valyl-L-proline, which still contained ca 10 g L-valine and ca 300 g potassium chloride was set to pH 6.4 and concentrated in a vacuum to a thick crystal paste. 1 1 n-butanol was then added and the mixture was heated for 2 hours in the water separator. According to HPLC the mixture then contained 3% of the dipeptide. After cooling 800 ml water was added and the phases were separated. The organic phase was washed again with 200 ml water and concentrated in a vacuum. The remaining crystal suspension was stirred up with ethyl acetate and the solid was filtered off. 70.5 g (58% of theoretical) cyclo[L-valyl-L-prolyl] with an HPLC purity of >99% and an [α]D/20 of −164.3° (c=1, n-butanol) was obtained.

Production of Cyclo[L-leucyl-L-prolyl]

1350 ml of an aqueous solution of 145 g L-leucyl-L-proline, which still contained ca 7 g L-leucine and ca 225 g potassium chloride was set to pH 4.5 and concentrated in a vacuum to a thick crystal paste. 1 1 n-butanol was then added and the mixture was heated for 0.5 hours in the water separator. According to HPLC, the mixture then still contained 3% of the dipeptide. After cooling, 500 ml water was added and the phases were separated. The organic phase was washed again with 100 ml water and concentrated in a vacuum. The remaining crystal suspension was stirred up with ethyl acetate and the solid was filtered off. 91.8 g (69% of theoretical) cyclo[L-leucyl-L-prolyl] with an HPLC purity of >99% and an [a]D/20 of −137.40 (c=1, n-Butanol) was obtained.

Production of cyclo[L-isoleucyl-L-prolyl]

2030 g of an aqueous solution of 199 g L-isoleucyl-L-proline, which still contained ca 7 g L-isoleucine and ca 300 g potassium chloride was set to pH 6.4 and concentrated in a vacuum to a thick crystal paste. 1 1 n-butanol was then added and the mixture was heated for 1 hour in the water separator. According to HPLC, the mixture then still contained 1% of the dipeptide. After cooling, 500 ml water was added and the phases were separated. The organic phase was washed again with 100 ml water and concentrated in a vacuum. The remaining crystal suspension was stirred up with MtBE and the solid was filtered off. 126.3 g (70% of theoretical) cyclo[L-isoleucyl-L-prolyl] with an HPLC purity of >99% and an [α]_{D/20} of −105.1° (c=1, n-butanol) was obtained.

Production of cyclo[ε-trifluoroacetyl-L-lysyl-ε-trifluoroacetyl-L-lysyl]

500 ml of a butanolic solution of 21 g ε-trifluoroacetyl-L-lysyl-ε-trifluoroacetyl-L-lysine hydrochloride was set to pH 6 with 50% sodium hydroxide solution and heated in the water separator for 2 hours. According to HPLC analysis, 57% of the dipeptide had then been cyclised to DKP. The solid deposited after cooling is filtered off and dried. 8.0 cyclo[ε-trifluoroacetyl-L-lysyl-ε-trifluoroacetyl-L-lysyl] was obtained.

¹H-NMR (d₆-DMSO): 1.30 (m, 4H), 1.48 (m, 4H), 1.67 (m, 4H), 3.17 (m, 4H), 3.80 (m, 2H), 8.13 (s, 2H), 9.43 (s, 2H).

1. Process for the production of 2,5-diketopiperazines of general formula I, 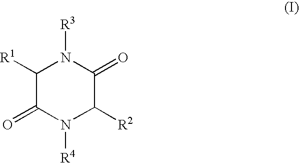
in which R¹, R² independently of each other represent H, (C₁-C₈)-alkyl, (C₂-C₈)-alkenyl, (C₂-C₈)-alkinyl, (C₁-C₈)-alkoxy, (C₃-C₈)-cycloalkyl, (C₆-C₁₈)-aryl, (C₇-C₁₉)-aralkyl, (C₃-C₁₈)-heteroaryl, (C₄-C₁₉)-heteroaralkyl, ((C₁-C₈)-alkyl)₁₋₃-(C₃-C₈)-cycloalkyl, ((C₁-C₈)-alkyl)₁₋₃-(C₆-C₁₈)-aryl, ((C₁-C₈)-alkyl)₁₋₃-(C₃-C₁₈)-heteroaryl, or the side chain group of an α-amino acid,
R³, R⁴ independently of each other represent H, (C₁-C₈)-alkyl, (C₂-C₈)-alkenyl, (C₂-C₈)-alkinyl, (C₁-C₈)-acyl, (C₃-C₈)-cycloalkyl, (C₆-C₁₈)-aryl, (C₇-C₁₉)-aralkyl, (C₃-C₁₈)-heteroaryl, (C₄-C₁₉)-heteroaralkyl, ((C₁-C₈)-alkyl)₁₋₃-(C₃-C₈)-cycloalkyl, ((C₁-C₈)-alkyl)₁₋₃-(C₆-C₁₈)-aryl, ((C₁-C₈)-alkyl) ₁₋₃-(C₃-C₁₈)-heteroaryl, or R¹ and R³ and/or R² and R⁴ form a ring via a (C₂-C₈)-alkylene unit,
by heating dipeptides of general formula II 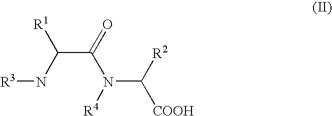
in which R¹, R², R³, R⁴ have the meaning given above, in an organic solvent whilst removing water by distillation.

2. Process according to claim 1,
characterised in that
an organic solvent is used, which forms an azeotrope with water.

3. Process according to claim 2,
characterised in that
n-butanol is used as the organic solvent.

4. Process according to one or more of the preceding claims,
characterised in that
the reaction is carried out at temperatures of 80-150° C.

5. Process according to one or more of the preceding claims,
characterised in that
the reaction is carried out in a pH range of 3 to 7.

6. Process according to one or more of the preceding claims,
characterised in that
the dipeptides of formula (II) are used in the reaction in the form of an aqueous solution.

7. 2,5-diketopiperazines of general formula III, 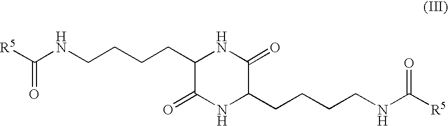
in which R⁵ represents H or trifluoromethyl.

8. 2,5-diketopiperazine according to claim 7,
characterised in that
it is present in the (S,S) configuration.

9. Dipeptides of general formula (IV), 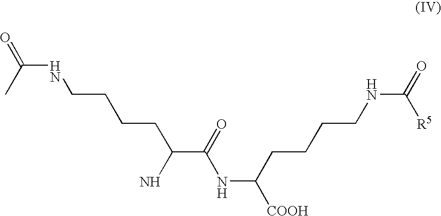
in which R⁵ represents H or trifluoromethyl.

10. Dipeptide according to claim 9,
characterised in that
it is present in the (S,S) configuration.

11. Use of the compounds according to one or more of claims 7 to 10 for the production of cyclo[Lys-Lys].

12. Use of the compounds produced according to claim 1 in the synthesis of bio-active compounds.
